(19) United States
(12) Patent Application Publication
(10) Pub. No.: US 2003/0135132 A1
(43) Pub. Date: Jul. 17, 2003

(54) Method and device for monitoring a muscle or group of muscles

(76) Inventor: Robert Leonard Krullaards, Leidschendam (NL)

Correspondence Address: Townsend & Townsend & Crew, Eighth Floor, Two Embarcadero Center, San Francisco, CA, 94111, US

(21) Appl. No.: 10/168,725

(22) PCT Filed: Dec. 22, 2000
(86) PCT No.: PCT/NL00/00957

(30) Foreign Application Priority Data
Dec. 22, 1999 (NL) 1013921

(51) Int. Cl. A61B 5/103

(52) U.S. Cl. 600/587

(57) ABSTRACT

The invention relates to a method for monitoring a muscle or group of muscles, comprising the steps of measuring the load on a muscle or group of muscles, comparing the load with a first threshold value and generating a warning signal when the measured load exceeds the first threshold value. The invention also relates to a device for monitoring a muscle or group of muscles, comprising a sensor for measuring the load on the muscle or group of muscles, comparing means connected to the sensor for comparing the measured load with the determined first threshold value and signalling means connected to the comparing means for generating a warning signal when the measured value exceeds a threshold value. This enables the use of a feedback system whereby the attention of the user is drawn to a situation in which he or she is moving towards a determined position possibly resulting in muscular problems.

In recent times many problems have arisen in relation to the results of tensions prevailing in human muscles. “RSI” in particular has received much media attention in recent times. The problem of “tennis elbows” has however been known for much longer.

These problems are caused by maintaining directly or indirectly too high a muscle tension in the arm for too long, which causes too high a tension in the neck-shoulder area, whereby neurological and vascular symptoms occur.

These problems can also be brought about by other causes, such as an incorrect posture, stress, writing rapidly and without a break, an inappropriate arrangement of the working surface or the desk or other causes forcing the neck or shoulders into too high a position, or holding them in such a position.

In order to avoid these problems the invention provides a method for monitoring a muscle or group of muscles, comprising the steps of measuring the load on a muscle or group of muscles, comparing the load with a first threshold value and generating a warning when the measured load exceeds the first threshold value.

This enables the use of a feedback system whereby the attention of the user is drawn to a situation in which he or she is moving towards a determined position possibly resulting in muscular problems.

Such a method can be used both preventively and therapeutically. The method can also be used to enhance comfort.

It is also pointed out that the muscle or group of muscles of which the tension or displacement is measured does not have to be the same muscle or group of muscles in which the possible symptoms occur; it is quite well possible for tension or displacement in a first muscle be caused by another muscle or by general tensions not directly related to muscles.

The invention further relates to a device for monitoring a muscle or group of muscles, comprising a sensor for measuring the load on the muscle or group of muscles, comparing means connected to the sensor for comparing the measured load with the determined first threshold value and signalling means connected to the comparing means for generating a warning signal when the measured value exceeds a threshold value.

It is the intention that such a device be used in performing the method according to the invention.

According to a first preferred embodiment the method comprises the measure that the time is measured from the moment the threshold value is exceeded, and a second warning signal is generated when the measured time exceeds a threshold value.

The invention also provides a device which is characterized in that the device comprises time-measuring means connected to the sensor and the comparing means for measuring the time from the moment the threshold value is exceeded, and that the signalling means are adapted to generate a second warning signal when the measured time exceeds a threshold value.

A system is hereby obtained wherein a warning signal is only generated when a position which might lead to muscle problems is held for a determined time.

This prevents excessive use of warning signals, for instance when changing position and the like.

According to another preferred embodiment a part of the body connected to the muscle or group of muscles for monitoring is monitored and a warning signal is generated when the monitored part of the body performs a movement.

An additional signal is hereby obtained, for instance when a determined part of the body is caused to move repeatedly.

The sensor is preferably formed by a movement sensor or pressure sensor which is placed outside the body, but which is intended for mechanical connection to the body.

Medical intervention is hereby avoided, while a good coupling between body and sensor is still obtained.

So as to enhance the comfort of the device, the sensor is fitted with a wireless signal transmission path between the sensor and the signalling means.

Other attractive preferred embodiments are stated in the remaining sub-claims.

The invention is now elucidated on the basis of an embodiment, wherein reference is made to the annexed drawing in which corresponding components are designated with the same reference numbers and in which:

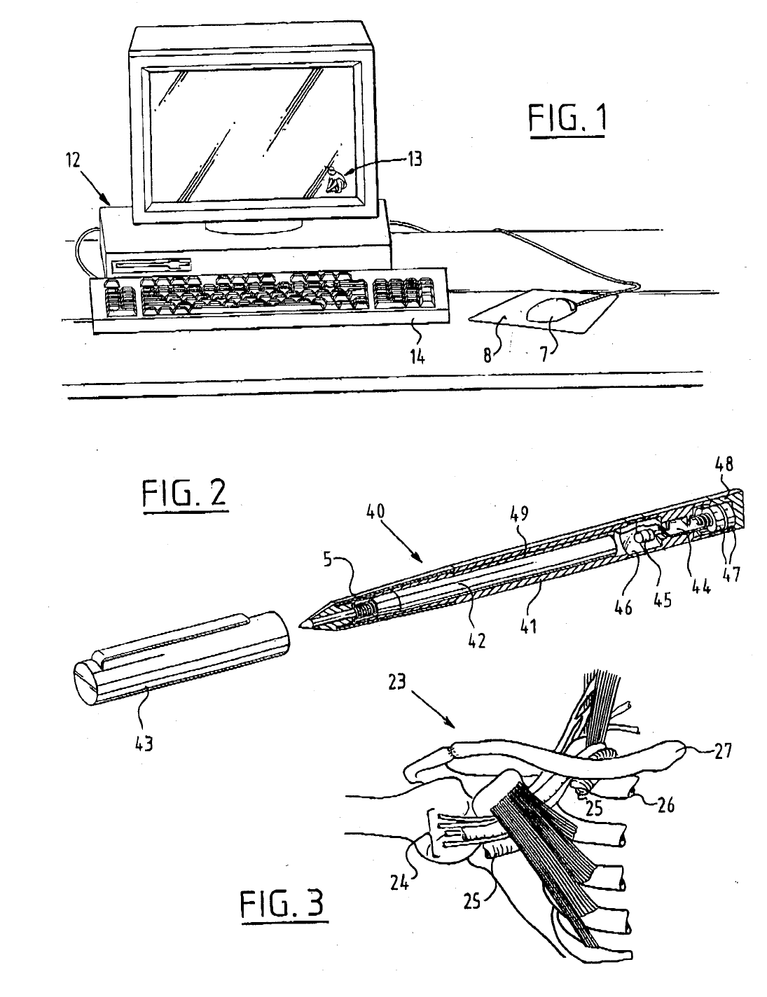

FIG. 1 is a schematic perspective view of a workplace with a computer which is provided with a device according to the invention,

FIG. 2 is a perspective cross-sectional view of a ballpoint provided with sensors according to the invention,

FIG. 3 is a schematic perspective view of a part of the human body,

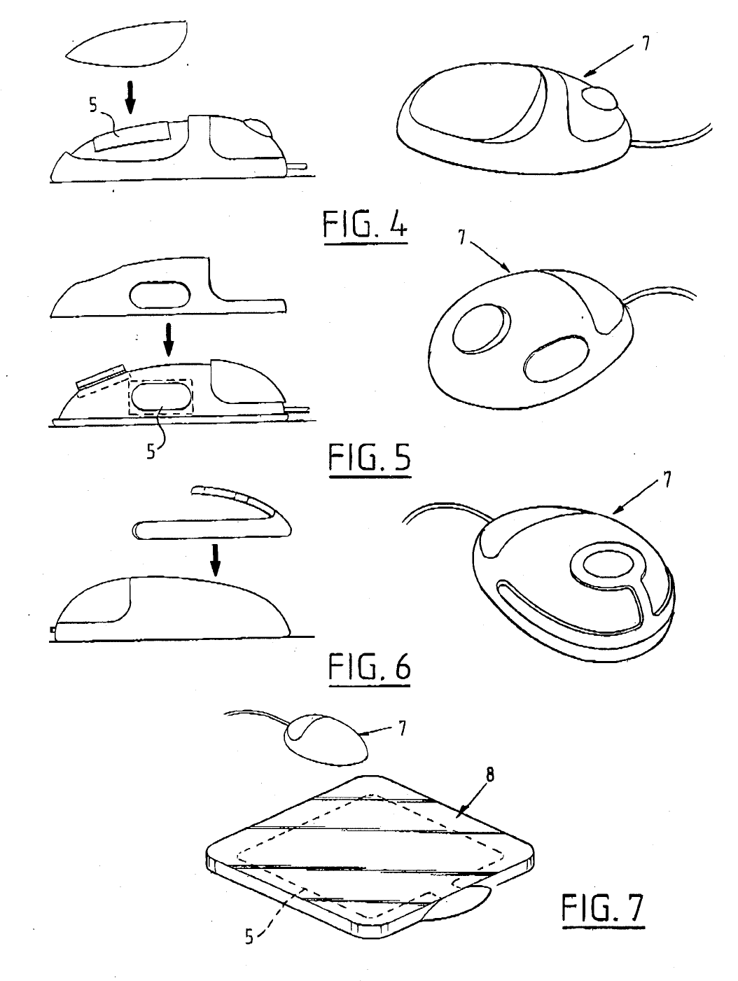

FIGS. 4, 5 and 6 show perspective views of computer mice into which the device according to the invention is integrated,

FIG. 7 is a perspective view of a mouse mat with the device therein,

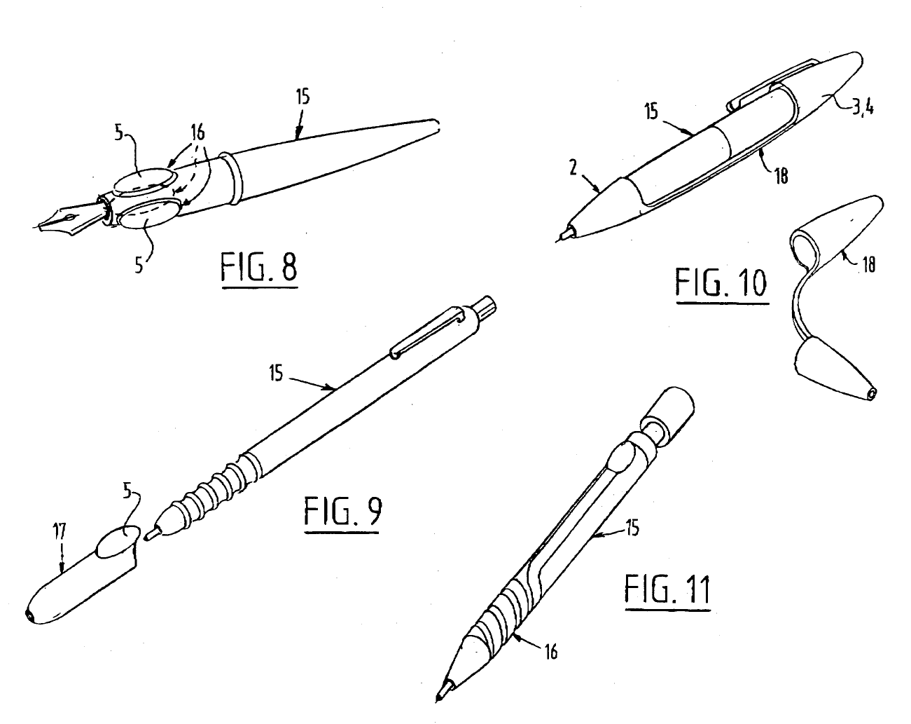

FIG. 8 shows the application of a device for preventing RSI according to the invention in a writing instrument,

FIGS. 9, 10 and 11 show alternative embodiments of the device according to the invention arranged on a writing instrument,

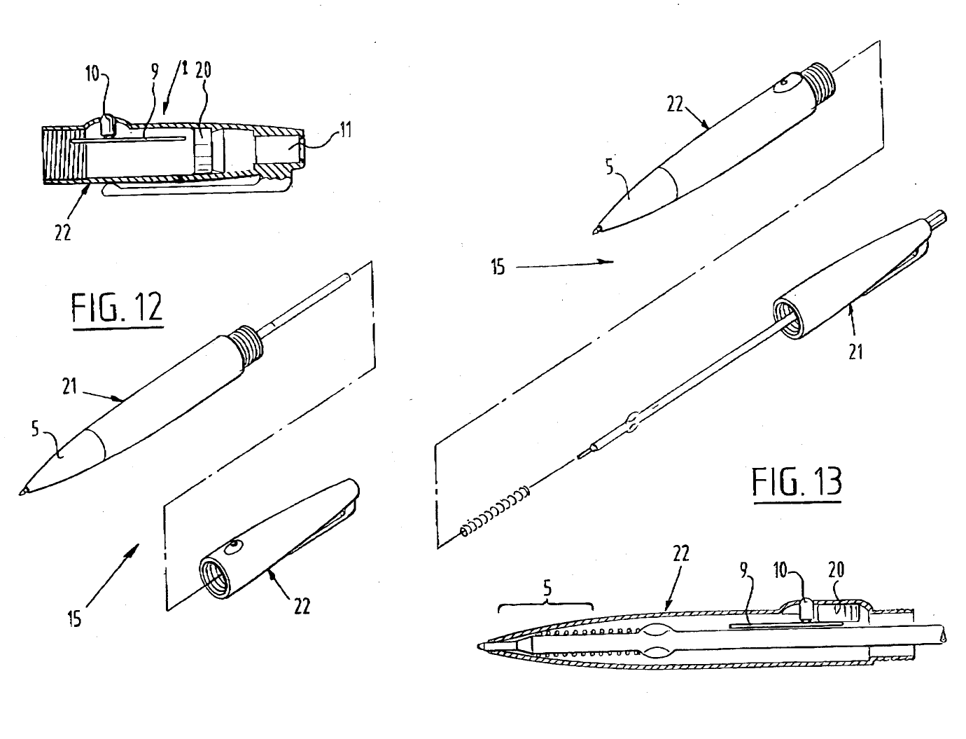

FIGS. 12 and 13 show two embodiments of the device according to the invention which are received in a writing instrument,

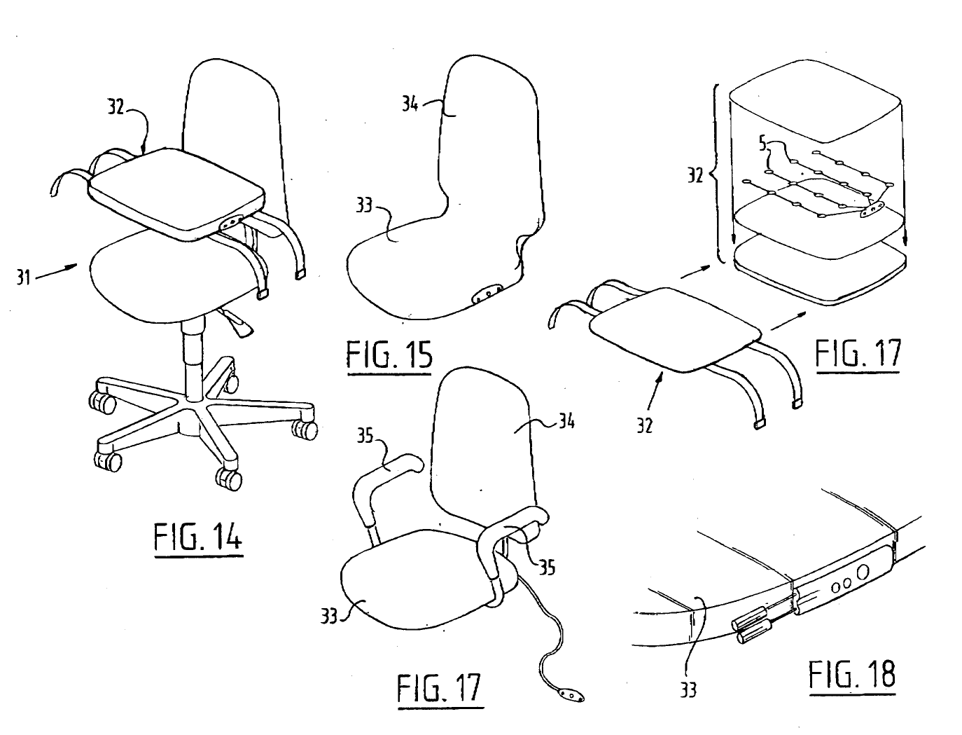

FIGS. 14, 15 and 16 show different options for devices according to the invention arranged in seat cushions,

FIG. 17 shows the structure of a cushion having therein a device according to the invention,

FIG. 18 shows a corresponding operating part of the device,

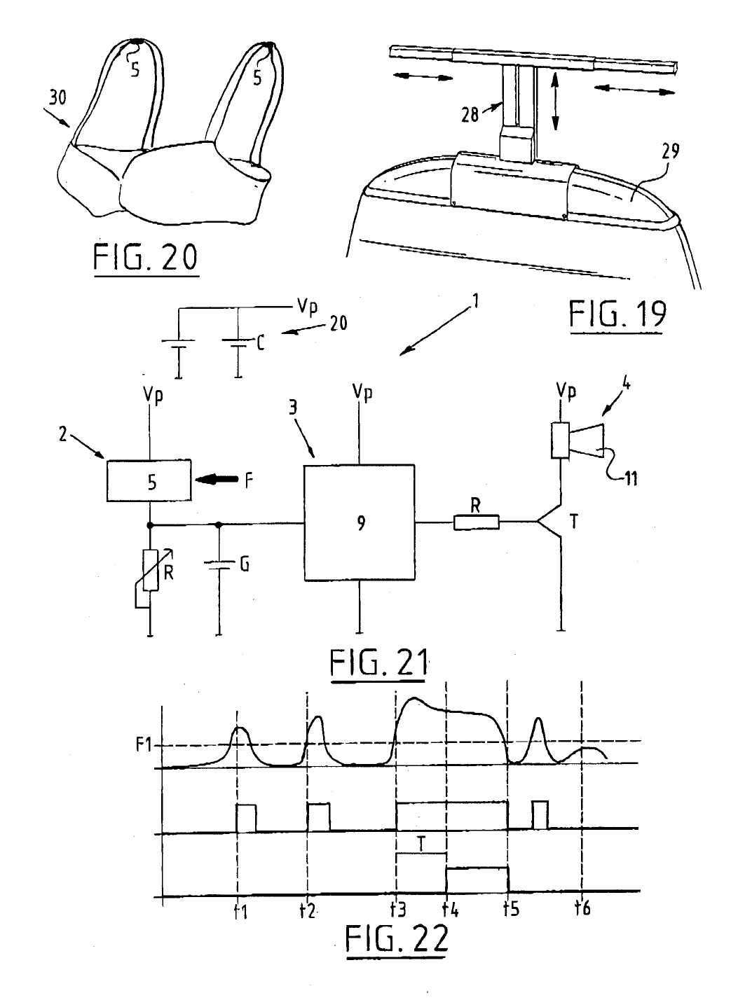

FIG. 19 shows an alternative embodiment of the device which is adapted to detect a movement of a body part connected to the relevant muscle, for instance the shoulder and which is connected to a piece of furniture,

FIG. 20 shows a variant of the device for detecting the movement of the shoulder which is arranged in an article of clothing,

FIG. 21 shows a simplified circuit diagram of the device according to the invention, and

FIG. 22 is a diagram of the progression of the load in time and the operation of the device resulting therefrom.

A device 1 for preventing RSI by avoiding overload of muscles comprises means 2 for measuring a load on the muscles, means 3 connected to load measuring means 2 for comparing the measured load with a determined first threshold value, and means 4 connected to comparing means 3 for generating a warning signal when the measured load exceeds the first threshold value. The function of signal generating means 4 does not otherwise have to be limited to generating a warning signal in the case of too high a muscle load, but these means can also be adapted to generate a release signal when the muscle load once again drops below the threshold value.

Load measuring means 2 can herein directly measure the load exerted by the muscles, but it is also possible for these measuring means to measure the load indirectly by detecting a movement of a part of the body resulting from the load on the relevant muscles.

In the first case the load measuring means 2 can comprise one or more sensors 5 which are connected to a tool 6 which is operated by the muscles. As sensor 5 can be used for instance a pressure sensor with which a pressure force or grasping force of the muscles can be measured. The use of a strain sensor to measure a tensile force can however also be envisaged.

Sensor 5 can be arranged in a computer mouse 7 (FIGS. 4, 5 and 6) or in a mouse mat 8 (FIG. 8).

As stated, sensor 5 is connected to comparing means 3, which can for instance take the form of a suitably programmed electronic circuit. This electronic circuit can be embodied as integrated circuit in the form of a microchip 9 (FIG. 21). On this chip 9 can also be accommodated the signal generating means 4 which can likewise be formed by a programmed electronic circuit. Sensor 5 and microchip 9 are herein connected to a power supply 20, for instance a battery.

The signal generating means 4 can further comprise a light source 10 and/or a sound source 11. Light source 10 can herein be a LED, which is relatively small and requires little energy.

The principle of the method according to the invention can be seen in FIG. 22. At points in time t1, t2 and t3 the sensor 5 is pressed in. As soon as the force exerted by the muscles on sensor 5 is greater than the threshold value F1, a clock is started internally in microchip 8. If the load falls below the threshold value again within a predetermined time T, nothing will happen, as can be seen at the points in time t1 and t2. If the force exerted on sensor 5 remains greater than the threshold value F1 for longer than the time T, a warning signal will be generated after this time T has elapsed for as long as threshold value F1 remains exceeded. For an optimal result the threshold value of the force F1 and the delay time T can be adjusted independently of each other.

When the load measuring means are accommodated in a mouse 7 or mouse mat 8, the rest of the device can also be accommodated therein, although it is also possible to include the comparing means 3 and signal generating means 4 in the form of an application program in a computer 12 connected to mouse 7. Computer 12 can therefore be programmed here to log the number of touches on a keyboard 14 connected to the computer, which may also form an indication of imminent RSI symptoms.

In the case computer 12 is used, the signals generated by signal generating means 4 can take the form of an animation. A cartoon character 13 can for instance be shown herein, the facial expression or even the general state of which forms an indication for the presence or absence of muscle tensions which involve the risk of RSI.

Computer 12 can herein be programmed such that the facial expression or state of the FIG. 13 changes gradually as the measured muscle tension increases or decreases. The computer can further be programmed such that the FIG. 13 gives information on the basis of which the user can adjust the load on his muscles, for instance by changing working posture or by temporarily interrupting his activity.

In order to encourage users to adopt a good working posture and good working habits, computer 12 can be programmed to keep a score, which incorporates the frequency of (imminent) overload of the muscles. This score can be displayed on the relevant computer, wherein the user can then try to improve his score, while it is also possible to mutually connect a number of computers 12 in a network, which opens up the possibility of an “RSI-competition”.

In addition to the comnputer mouse, writing instruments are also a source of RSI symptoms. In an alternative embodiment of the invention the sensors 5 are therefore mounted on a writing instrument 15 (FIG. 8). Each sensor can herein be integrated with a comparing circuit and signal generating circuit in very small form to form a warning unit 16. This warning unit 16 can also be releasably connected to writing instrument 15. In that case the unit for instance takes the form of a single sleeve 17 (FIG. 9) or dual sleeve 18 (FIG. 10) which can be pushed or clamped round writing instrument 15. In the case of the dual sleeve the comparing means 3 and signal generating means 4 are then accommodated in the rear compartment and the load measuring means 2 in the front one. Warning unit 16 could also take a self-adhesive form and for instance be wrapped round the writing instrument.

The different components of device 1 could also be received in the body 19 of writing instrument 15. A part 21 of writing instrument 15 can herein be of conventional structure, while device 1 is accommodated in the remaining part 22 (FIG. 12, 13).

In the ballpoint pen 40 shown In FIG. 2 a part of housing 41 is provided with a pressure sensor 5. Pressure sensor 5 is connected via two wires 49 to a circuit 44. Circuit 44 is powered by batteries 47 and a LED 45 is connected to circuit 44.

The ballpoint pen 40 is further provided with an ink cartridge 42. Because the present embodiment of the invention prevents the use of a clicking mechanism, the ballpoint pen can be closed by a cap 43. Further arranged is a transparent element 46, in a recess of which the LED 45 is placed. The space for the batteries is closed off by a cover 48.

Instead of a direct measurement of the load on the muscles, the movement of a part of the body connected to the muscles can, as stated, also be detected. It is possible for instance to choose the shoulder 23 for this purpose, which is pulled up when there is too great a strain on the arm muscles, wherein particular nerve-paths 24 and/or blood vessels 25 are irritated or pinched by the first rib 26 and the collar bone 27 (FIG. 3). The movement of shoulder 23 can be detected by an adjustable bracket 28 which rests on the shoulder, is adjustable in height and width and which can be mounted on the back 29 of a seat (FIG. 19). A movement of the shoulder can herein result in a displacement of bracket 28, which can be detected by a movement sensor (not shown here). It is also possible to derive the movement of shoulder 23 from a force which is exerted by the shoulder on an article of clothing 30. For this purpose a pressure sensor 5 can be arranged in this article of clothing 30, for instance a bra.

The detected movement of the shoulder is again compared with a threshold value by comparing means and results in a warning signal being generated when this value is exceeded,

Instead of the movement of shoulder 23 the pressure exerted by the body of a user on different parts of a seat 31 could also be taken as a measure for the muscle tension and the risk of RSI. Pressure sensors 5 can be received for this purpose in a separate cushion 32 which is fixed on seat 31 (FIGS. 14, 17), or in the actual seat part 33, seat back 34 and/or arm rests 35 of seat 31 itself (FIGS. 15, 16).

The invention thus makes it possible with relatively simple means to provide a warning when, as a result of for instance an incorrect working position or too great a number of repetitive actions, the danger of RSI is created, so that timely corrective measures can be taken.

Although the invention is described and shown above on the basis of a number of embodiments, it is not limited thereto. Many variations are possible both in the embodiment and placing of the load measuring means and in the manner in which the measured quantities are processed and signalled. The scope of the invention is defined solely by the appended claims.

1. Method for monitoring a muscle or group of muscles, comprising the steps of measuring the load on a muscle or group of muscles, comparing the load with a first threshold value and generating a warning signal when the measured load exceeds the first threshold value.

2. Method as claimed in claim l, characterized in that the time is measured from the moment the threshold value is exceeded, and a second warning signal is generated when the measured time exceeds a threshold value.

3. Method as claimed in claim 1 or 2, characterized in that a part of the body connected to the muscle or group of muscles for monitoring is monitored and a third warning signal is generated when the monitored part of the body performs a movement.

4. Method as claimed in claim 1, 2 or 3, characterized in that the load on the muscle or group of muscles is measured by an element placed outside the body.

5. Method as claimed in any of the foregoing claims, characterized in that the load on more than one muscle or group of muscles is detected.

6. Method as claimed in claim 5, characterized in that signals from more than one muscle or group of muscles are fed to a collective signalling device.

7. Device for monitoring a muscle or group of muscles, comprising a sensor for measuring the load on the muscle or group of muscles, comparing means connected to the sensor for comparing the measured load with the determined first threshold value and signalling means connected to the comparing means for generating a warning signal when the measured value exceeds a threshold value.

8. Device as claimed in claim 7, characterized in that the device comprises time-measuring means connected to the sensor and the comparing means for measuring the time from the moment the threshold value is exceeded, and that the signalling means are adapted to generate a second warning signal when the measured time exceeds a threshold value.

9. Device as claimed in claim 7 or 8, characterized in that the sensor is formed by a movement sensor or by a pressure sensor.

10. Device as claimed in any of the claims 7-9, characterized in that the sensor is adapted to be used in mechanical contact with the body.

11. Device as claimed in claim 7, 8, 9 or 10, characterized in that a wireless signal transmission path is arranged between the sensor and the signalling means.

12. Device as claimed in any of the claims 7-11, characterized in that the signalling means are adapted to receive signals from more than one sensor.

13. Device as claimed in claim 12, characterized in that the sensors from which the signalling means receive the signals are placed to sense the load of muscles or groups of muscles belonging to a person.

14. Device as claimed in any of the claims 11-13, characterized in that the signalling means are arranged in a digital computer programmed for this purpose.

15. Device as claimed in any of the claims 7-14, characterized in that the sensor is arranged in an object to be used by hand, such as a writing instrument, a mouse, a stroke-making element for use in sport, or a tool.

16. Device as claimed in claim 15, characterized in that the comparing means and the signalling means are arranged in the object to be used by hand.

17. Device as claimed in any of the claims 7-14, characterized in that the sensor is adapted to be in mechanical connection with the shoulders, the back, the seat or the soles of the feet.
